(19) United States
(12) Patent Application Publication
(10) Pub. No.: US 2019/0128870 A1
(43) Pub. Date: May 2, 2019

(54) Diagnostic Methods For Patient Specific Therapeutic Decision Making In Cancer Care

(71) Applicant: Humeltis, Pecs (HU)

(72) Inventors: Judit Erzsebet PONGRACZ, Hosszuheteny (HU); Judit RAPP, Pecs (HU); Evelin RACZ, Pecs (HU)

(21) Appl. No.: 16/091,434

(22) PCT Filed: Apr. 4, 2017
(86) PCT No.: PCT/EP2017/058034

§ 371 Date: Oct. 4, 2018

(30) Foreign Application Priority Data
Apr. 4, 2016 (GB) 1605759.8

(51) Int. Cl. G01N 33/50; C12N 5/09

(52) U.S. Cl. CPC G01N 33/5011; C12N 2513/00; C12N 2503/02; C12N 5/0693

(57) ABSTRACT

The present invention relates to a 3-Dimensional (3D) tissue culture aggregate of cells derived from a neoplastic tissue sample, wherein ≤30% of total number cells are cells capable of interfering with re-aggregation. It also relates to a method of making such a 3D aggregate and a method for assessing the effectiveness of an anti-neoplasm treatment by measuring the effect of said treatment on the viability of a three dimensional (3D) neoplasm tissue culture aggregate.

The present application relates to a 3D aggregate of tumour cells which forms without an artificial scaffold, methods of making these 3D aggregate of tumour cells and a method of assessing sensitivity of a tumour cell to a therapeutic agent, utilising said 3D aggregate of tumour cells.

The overall survival of patients suffering from proliferative diseases depends on the stage at the time of the diagnosis. For example, 5-year survival rate of NSCLC varies from 73% in early detection (stage IA) to 3.7% at advanced metastatic disease. At early stages of NSCLC surgery and chemotherapy are still the choice of first line treatment, although targeted molecular therapies are now more widely included in the treatment regimen. Targeted therapies that can extend progression free and overall survival are only available to a fraction of patients, as such approaches require the presence of mutations or amplifications of one of the following genes: the epidermal growth factor receptor (EGFR), echinoderm microtubule-associated protein-like 4-anaplastic lymphoma kinase (EML4-ALK) kinase translocation, KRAS and PI3KCA, which only affect a relatively small percentage of patients.

Unfortunately, many patients present at advanced or even metastatic stage of their diseases where surgical resection is not an option. Adjuvant cisplatin based therapy can increase the survival rates in all stages but chemotherapy resistance and disease recurrence remain major issues. Metastatic non-small cell lung cancers for example, treatment is frequently based on the combination therapies of cisplatin or carboplatin with drugs such as paclitaxel, docetaxel, gemcitabine and vinorelbine which can increase efficacy compared to single agent platinum therapy. Although the use of immune modulators (e.g. Nivolumab) have become a promising route to effectively halt disease progression, their application in fast progressing tumour types require further analysis. Therefore a clinician can only rely on his or her experience to choose from the available drug panel.

Currently, the effectiveness of a selected combination therapy cannot be predicted. Using 3D tissue cultures built from individual tumours can change current trends and can aid clinical decision making.

Personalized medicine (or precision medicine, PM) proposes patient specific customization of treatment tailored to the needs of an individual patient. To achieve this aim various diagnostic tests are employed for selecting appropriate and optimal therapies based on the context of a patient's genetic makeup or other molecular or cellular characteristics.

In a great variety of diseases PM is being used successfully. Unfortunately, proliferative diseases, especially various cancers are not amongst the clear-cut success stories despite repeated claims stating otherwise. Especially so in late, metastatic stages of proliferative diseases when only palliative care is offered to cancer sufferers with no hope for effective treatment.

Several attempts have been made to use primary, surgical samples to test drug sensitivity of individual patients. Out-growth cultures, where the proliferative ability of tumour cells to grow under cell culture conditions, are the best known. Test systems of “out-growth” cancer cultures, however, face a vast number of difficulties. Amongst others, such cultures are lacking the complexity of individual tumours and therefore unable to correctly represent the tumour and therefore predict the responses to specific drugs. Since the recognition that the tumour microenvironment where the cell-cell interactions are just as important as the mutations in the cancerous epithelial cells, a large number of cellular systems have been developed to re-create the three-dimensional tumour microenvironment.

It has been recognized that, to avoid losing the complex structure and molecular microenvironment of an individual tumour, three-dimensional tumour cultures using cells of the patient should be created and drug sensitivity tests should be performed in such cultures [Edmondson, Rasheena et al., “Three-Dimensional Cell Culture Systems and Their Applications in Drug Discovery and Cell-Based Biosensors.” Assay Drug Dev Technol. 2014 May 1; 12(4): 207-218.]

Presently, there is a strive towards personalized medicine and targeted therapy and to create the most appropriate in vitro model that closely mimics the in vivo tumour microenvironment. Currently there is a mix of traditional 3D platforms and emerging technologies which rely on the advantage of polymer matrices to recreate porous structures for cell maintenance. [Caicedo-Carvajal C E et al. “Three-Dimensional Cell Culture Models for Biomarker Discoveries and Cancer Research”, Translational Medic S1: 005, Feb. 13, 2012].

A 2013 review of in vitro three-dimensional cancer culture models provides a highly relevant list of methods and technologies to develop three-dimensional cancer models [Asghar, Waseem et al, “In Vitro Three-Dimensional Cancer Culture Models.” Cancer Targeted Drug Delivery, pp 635-665, 10 Jul. 2013].

These methods include embedded and overlay cell cultures wherein cells are present in gelled artificial extracellular matrix (ECM) either embedded (wherein cells are suspended into the basement membrane) or, in an overlay culture, where a basement membrane is applied to the surface of a substrate and forms a thin hydrogel coating. In practice scaffolds are usually applied to provide a natural-like matrix environment of the cells. These scaffold types are discussed in detail by Asghar, Waseem et al. As a future perspective, it is noted nevertheless, that Scaffold-free 3D micro-tissue models are considered more organotypic and compatible with high-throughput technologies.

It appears, however, that the era of high throughput drug sensitivity testing using scaffold-free spherical tumour microtissues has not yet come [Drewitz M, Helbling M, Fried N, Bieri M, Moritz W, Lichtenberg J, Kelm J M (2011) Towards automated production and drug sensitivity testing using scaffold-free spherical tumour microtissues. Biotechnol J 6(12):1488-1496

That said, a large amount of knowledge has accumulated in the prior art regarding three dimensional cancer micro-tissue models using patient-derived cells.As mentioned above, a large number of prior art solutions apply some kind of extracellular matrix.

WO2015/073724 describes a method of testing proliferative responses of a drug on patient-derived tumour cells; the method comprising, obtaining cells from biopsy or tumour resection material; culturing the cells on a 3D extracellular matrix (ECM); treating the cells in ECM with a drug; subjecting the treated cells to high-content (HC) imaging; and evaluating the HC imaging of the treated cells; thereby testing the proliferative responses of the drug on the patient-derived tumour cells. As mentioned, the cells are subcultured in 3D on 1:20 ECM.

WO2014/200997 provides a method for producing an isolated, unencapsulated, three dimensional organotypic cell culture product wherein harvested cells are resuspended in a naturally derived gel matrix, a gelled three-dimensional cell matrix is formed in a hydrophobic solution from which the organotypic cell culture is isolated and cultured within the 3D gel matrix. All the experimental results are obtained with cell lines, as opposed to primary cells or tissues. The application of a hydrophobic solvent and the use of a gel matrix means this system may not be reliable, in particular as a high throughput screen (HTS).

WO2015/196012 describes a method wherein each individual cell line applied is marked with a nucleic acid sequence. A cultured pool of the cell lines is subjected to treatment e.g. by chemotherapy and the resulting pool of cell lines is analyzed via these labels.

US2013/012404 and US2014/128272 provide a cancer tissue derived cell mass by isolating a tumour xenograft, subjecting it to enzymatic treatment and a cell strainer, removing single cells, small cell masses and debris, centrifuging several times before culture. The culture is suitable for studying the dormant state of cancer cells. As the spheres can be frozen they can be stored for further study e.g. sequencing.

The primary focus of the assay described in US2014/336282 is the functional ability of the cancer cells to invade. The molecular phenotype is the description of the cells that share a functional attribute. The authors have defined a specific molecular signature, the basal leader signature (keratin 14+, p63+, P-cadherin+ and smooth muscle cell actin-) that correlates with the most invasive subpopulation in mouse tumour models and with the cellular identity of micrometastases. This gene expression signature could be used to identify invasive subpopulations in sections from fixed tissue from archival human tumours.

Organoids were embedded in collagen gels or matrigel.

US2016/040132 describes potential methods of identifying a therapeutic agent for pancreatic cancer in an individual. The method comprises preparing a stromal bio-ink; preparing a tumour bio-ink; and bioprinting the stromal bio-ink and the tumour bio-ink such that the tumour bio-ink is encased in the stromal bio-ink and in contact with the stromal bio-ink on all sides. The stromal bio-ink comprises pancreatic stellate cells and endothelial cells and optionally a hydrogel; the tumour bio-ink comprises primary pancreatic cancer cells from the individual. The deposited bio-ink is matured in a cell culture media to allow the cells to cohere to form a three-dimensional, engineered, pancreatic tumour model. This maturing takes typically a few days, e.g. 5 to 10 days. A candidate therapeutic agent is applied to the pancreatic tumour model; and the viability of the pancreatic cancer cells measured. A therapeutic agent is selected for the individual based on the measured viability of the pancreatic cancer cells.

In a number of prior art solutions the aim is to select or outgrow the most aggressively proliferating cells or the most invasive cancer cells. Alternatively a population with a specific molecular phenotype is isolated and then compared to unsorted or alternatively sorted populations. Unfortunately, such systems are still deprived of important non-neoplastic cells, e.g. the patient's tumour specific immune cells, therefore immune modulatory effects of recent cancer drugs cannot be explored.

Alternatively, in certain methods no selection of cell types were made but cells from the tumour samples were cultured usually applying an artificial scaffold.

The present inventors have applied a different approach to obtain three dimensional (3D) neoplasm tissue culture aggregates duly modelling or faithfully reflecting the composition of tumour, which are still suitable for HTS, as well as capable of being stored and reproduced.

The present inventors have surprisingly recognized that by reducing the relative ratio of cells capable of interfering with re-aggregation to tumour cells, then the formation of 3D tissue cultures from cells obtained from individual patients is possible in the absence of any artificial scaffold or extracellular matrix as a glue. Three dimensional (3D) neoplasm tissue culture aggregates can be prepared, which are suitable for testing anti-cancer treatment methods, if the ratio of cells capable of interfering with re-aggregation such as lymphoid cells (CD45+ cells) is reduced in an initial population of cells obtained from a tumour sample from a patient to be treated.

This reduction of the ratio of cells capable of interfering with re-aggregation, such as lymphoid cells (lymphocytes) allows the maintenance of an otherwise tumour-like composition wherein the cells are patient derived cells. If necessary, fibroblasts are added to provide an appropriate level of extracellular matrix (ECM) without adding an artificial scaffold.

The method of the present invention uses patient-derived cells so the aggregate formed can be used to select the most effective treatment. Anti-neoplasm compounds or treatments, such as chemotherapeutic agents, or combinations thereof can be tested, and those which reduce the tumour cell viability can be used to treat the patient. The aggregate is preferably free of any artificial scaffold.

In a first aspect the present invention relates to a 3-Dimensional (3D) tissue culture aggregate of cells derived from a neoplastic tissue sample wherein ≤30% of total number cells are cells capable of interfering with re-aggregation; wherein said aggregate does not contain an artificial scaffold.

Preferably said cells capable of interfering with re-aggregation are lymphoid cells e.g. lymphocytes. Preferably said cells capable of interfering with re-aggregation are CD45+ cells. More preferably, the cells capable of interfering with re-aggregation are CD45+ cells with lymphoid origin The number of cells capable of interfering with re-aggregation should be equal or lower than 30% of the total cell number/aggregate. Preferably the number of cells capable of interfering with re-aggregation cells should be between 5-20% of the total cell number/aggregate, for example 7-17%; or 10-15% of the total cell number/aggregate.

As used herein “cells capable of interfering with re-aggregation” refer to cells, which if present in sufficient quantity prevent the formation of a cell aggregate from patient derived tumour cells, preferably in the absence of an artificial scaffold or matrix. As some cells such as lymphoid cells can interfere with re-aggregation ability of the other cell types (epithelium, endothelium, fibroblast, smooth muscle cell) present, proportional reduction of such cells may be necessary to re-create individual tumours. Cells with lymphoid origin are commonly CD45+. Typically the cells capable of interfering with re-aggregation are CD45+ cells. The cells capable of interfering with re-aggregation may be lymphoid cells, preferably CD45+ lymphoid cells. Typically in cellular aggregates of the invention 30% but more than 5% of the total number of cells, preferably ≤25% or ≤20% are cells capable of interfering with re-aggregation.

A “neoplasm” or “cancer” is defined herein as a condition characterized by unregulated or uncontrolled proliferation of cells within a subject. The proliferation usually results in developing a lump or a mass of cells which is called a “tumour”. A “solid tumour” is a tumour which has a definite tissue structure and three dimensional shape. Tumours include carcinomas, myelomas,sarcomas such as glioblastomas, gliomas, Neuroblastoma, Medulloblastoma, adenocarcinomas, Osteosarcoma, liposarcomas, Mesothelioma, Hepatoma, hepatocellular carcinoma, Renal cell carcinoma; hypernephroma, Cholangiocarcinoma, and Melanoma.

Cancers includes kidney (renal), liver, brain, lung including small cell (SC/LC) lung cancer and non-small cell lung cancer (NSCLC), skin, bone, epithelial, intestinal, stomach, colon, mouth (oral), breast, prostate, vulval/vaginal, testicular, neuroendocrine, bladder, cervical, pancreatic, multiple myeloma, Waldenstrom macroglobulinemia, non-secretory myeloma, smoldering multiple myeloma, MGUS, light-chain myeloma, primary systemic amyloidosis, and light chain-deposition disease.

A cancer or neoplasm is considered herein as “malignant” if it has a tendency to result in a progressive worsening of the condition of the subject, i.e. has a deleterious effect in the subject and to potentially result in death. A cancer may also considered as malignant if the lump or mass of cells (e.g. a tumour) which develops initially appears or is diagnosed as not to be malignant, i.e. “benign” but (i) carry the risk of becoming malignant, or (ii) becomes malignant later in time.

A neoplastic tissue sample can be part or all of a tumour obtained via biopsy or tumour resection. The sample may be obtained from a primary solid tumor (regardless of origin) or metastatic tissues from lymph nodes and or other organs. Alternatively a neoplastic tissue sample may comprise accumulated fluids including pleural e.g. malignant pleural effusion (M PE) or malignant peritoneal effusion (ascites) fluids containing neoplastic cells together with other types of cells forming the neoplastic tissue.

The neoplastic tissue sample is obtained from a subject. A “subject” is understood herein as an animal, preferably a warm-blooded animal, a mammal or a human. Preferably the subject has been previously diagnosed as having cancer or a neoplasm. Preferably the subject is a patient. A “patient” is a subject who is or is intended to be under medical or veterinarian observation, supervision, diagnosis or treatment. More preferably the subject is the patient to whom treatment, including prophylactic treatment, has been or is to be provided.

As used herein, the term “treatment” of a condition or a patient having a neoplasm refers to any process, action, application, therapy, or the like, wherein the patient is under aid, in particular medical or veterinarian aid with the object of improving the patient's condition, either directly or indirectly. Treatment typically refers to the administration of an effective amount of an anti-neoplastic compound or composition, such as a chemotherapeutic agent. In a broader sense the term ‘treatment’ includes preventive treatment. In a narrower sense treatment is applied when at least one symptom, or at least a molecular marker, indicating the presence of the condition or the fact that onset of such a condition is imminent can be shown. If a condition is treated, it is preferably alleviated or improved i.e. its symptoms are reversed or at least further onset of the condition is prevented.

As used herein “artificial scaffold” refers herein to a scaffold or matrix which is a pre-formed scaffold integrated into the physical structure of the engineered tissue and which cannot be removed from the tissue without damage to or destruction of said tissue. Artificial scaffolds include polymer scaffolds, porous hydrogels, non-synthetic scaffolds like pre-formed extracellular matrices, dead cell layers, decellularized tissues etc.

Scaffold-free or “free of artificial scaffold” relates to a tissue wherein the scaffold is not an integral part of the engineered tissue at least at the time of its use. Preferably preparation of the aggregate of the invention does not require or use an artificial scaffold.

The present invention also provides a method for preparing a 3D tissue culture aggregate comprising:
(a) Preparing an adjusted cell population from a neoplastic tissue sample by reducing the number of cells capable of interfering with re-aggregation to ≤30% of total number cells; and
(b) Preparing a suspension culture comprising cells of said adjusted cell population, culture media and optionally fibroblasts; in the absence of an artificial scaffold.

The method may comprise the following steps:
1. Assessing the number of initial population of cells within a tumour tissue sample. The cell counts should reach a preferable number e.g. 2×10³ to 8×10⁵ cells
2. Adjusting the ratio of certain cell types to obtain an adjusted (processed) population
3. Preparing suspension cultures comprising cells of the adjusted (processed) population, a culture medium and optionally fibroblasts .
4. Optionally, cryopreserving the suspension culture.
5. Optionally, thawing the cryopreserved culture.
6. Providing initial aggregates from the suspension cultures.
7. Culturing the initial aggregates.

The cells within a neoplastic tissue sample can be dissociated. In addition, the samples can be treated, for example by washing, to reduce the number of red blood cells present.

Solid tumour samples can be reduced in size and undergo mechanical dissociation by cutting or mincing, for example using sterile scalpels. The cells in the tissue sample are dislocated according to known tumour dissociation methods, known in the art (see Langdon and Macleod (2004)” Essential Techniques of Cancer Cell Culture” Methods Mol Med.; 88:17-29.) such as the Miltenyi tumour dissociation method. A protocol suitable to the specific tumour type is utilised. Following dissociation, the cells sample can be washed if necessary to remove any red blood cells. Any red blood cells remaining can be lysed using methods known in the art, such as using a lysis buffer containing ammonium chloride. Once digestion is completed the number of cells present is counted prior to further processing.

MPE or ascites neoplastic tissue samples frequently contain large numbers of blood cells which are preferably removed using known methods. The samples are preferably treated with heparin.

For liquid neoplastic tissue sample such as MPE or ascites, the cells are sedimented, for example using centrifugation (e.g. 20 minutes at 300 g) to form a cell pellets. The supernatant can be removed and the pellet resuspended in an appropriate buffer e.g. phosphate-buffered saline optionally containing up to 20% of the cell free pleural or ascites fluid (i.e. supernatant). Mononuclear cells, such as white blood cells, can be separated from the cells within the suspension utilising well-known methods, such as Ficoll separation. The remaining cells can be isolated and counted prior to further processing.

The cellular composition of the tissue culture aggregate can be identified using surface cell marker analysis for example utilising flow cytometry. Surface cell markers can be identified using antibodies such as CD31-APC Cy7, CD44-FITC, CD45-PerCp, CD90-BV421, EpCam-APC.

The number of cells capable of interfering with re-aggregation may be reduced utilising a number of known techniques including immunological particle separation methods (such as magnetic manual or automated sedimentation, flow-through separation) and cell sorting separation methods such as flow cytometric automated cell sorting methods. These methods are well known to the person skilled in the art e.g. Immunology (2006) Luttman et al. Some suitable methods are described in the exemplary methods below such as the Miltenyi or EasySep methods. Preferably the number of cells capable of inhibiting reaggregation is less than 30% of the total number of cells in the initial cell suspension.

Preferably the cells capable of interfering with the aggregation are lymphoid cells. Preferably the cells capable of interfering with the aggregation are CD45+ cells, more preferably lymphoid CD45+ cells. The ratio of the cells capable of inhibiting reaggregation to other cell types within the initial cell suspension is preferably less than 30% of total number of cells. The ratio of lymphoid cells, preferably CD45+ cells is less than 30%, more preferably less than 25% or less than 20% in the adjusted population of cells. Preferably the number of lymphoid cells within the initial cell suspension is 5% or more.

The ratio of the CD45+ cells compared to other immune cells is preferably reduced. Preferably the number of cells capable of inhibiting reaggregation is less than 30% of the total number of cells in the initial cell suspension.

It may be necessary to add normal fibroblasts to the cells in order to form an aggregate, especially to create solid tumour from individual cells of MPE or ascites. The fibroblasts are usually obtained from the same tissue type as the tumour. For example, for cells of MPE or ascites, Normal Human Lung Fibroblasts are added. Preferably, the number of fibroblasts in the initial suspension culture is 5-50% total number of cells. For example the number of fibroblasts in the initial suspension culture may be at least 5%-50%, 10%-40% or 20%-30% total number of cells.

The initial cell suspension culture may comprise at least 2×10³ to 8×10⁵ cells from the adjusted population. Preferably the initial cell suspension culture comprises 2×10³ to 2×10⁴ ; or 10⁴ to 10⁵; or 5×10⁴ to 3×10⁵; or 5×10³ to 8×10⁵ cells, for example 5×10³ or 8×10³ or 10⁴ or 5×10⁴ or 8×10⁴ cells from the adjusted population.

The initial aggregates may be obtained from the suspension cultures by any well known method such as pelleting (e.g. by centrifugation), or the hanging drop method (e.g. Foty (2011) “A simple hanging drop cell culture protocol for generation of 3D spheroids” Journal of Visualized Experiments 6;(51)). Centrifugation can be carried out at 300 g to 1000 g, preferably at 400 g to 800 g or 500 to 700 g. Centrifugation can be carried out for 5 to 20 min, preferably from 5 to 15 min or 8 to 12 min, highly preferably at about 10 min. Centrifugation can be carried out at 0° C.—room temperature (up to 20° C.), preferably 4° C.-10° C. Centrifugation can be carried out at 0° C.-20° C., preferably 4° C. to 10° C.

Alternatively the initial aggregates may be obtained from suspension cultures by using matrix assisted tissue printing. (See Lijie Grace Zhang, John P Fisher, Kam Leong (2015) 3D Bioprinting and Nanotechnology in Tissue Engineering and Regenerative Medicine.)

The initial aggregates can be formed in the suspension cultures by using a scaffold (matrix). However, it is preferred that the aggregates are formed and cultured in the absence of an artificial scaffold or matrix.

The cells obtained from the tissue sample can be stored, preferably by cryopreservation. Thus, the tissue culture aggregates formed by the methods of the present application may be frozen and stored. The aggregates can then be thawed at a later stage. The viability of the aggregate is tested and if found to be positive, the cells can be used for further tests. For example, if an initial treatment is no longer effective or only partially effective a new treatment can be identified using the stored 3D aggregates

The invention provides a method for predicting and assessing the effectiveness of an anti-neoplasm treatment by testing the effect of treatment on three dimensional (3D) neoplasm tissue culture aggregates, preferably using an aggregate as defined herein or formed using a method as described herein.

The method comprises subjecting the 3D tissue culture aggregates to an anti-neoplasm treatment. For example, the aggregate can be contacted with a chemotherapeutic agents, or combination thereof. Following the treatment, the viability of the 3D neoplastic tissue culture aggregates is assessed. Results of the cell viability assays are compared to a control sample i.e. an aggregate which has not been treated with the anti-neoplasm treatment. Anti-neoplasm treatments identified as reducing cell viability can then be used to treat the patient.

“Anti-neoplasm treatment” refers to compounds or pharmaceutical formulations used to treat neoplastic conditions or cancers. These treatments include known chemotherapeutic agent and immunotherapies, and combinations thereof. Treatments may comprise a combination of more than one chemotherapeutic agent.

Chemotherapeutic or cytotoxic agents are known in the art. Suitable agents include Actinomycin, All-trans retinoic acid, Azacitidine, Azathioprine, Bleomycin, Bortezomib, Carboplatin, Capecitabine, Cisplatin, Chlorambucil, Cyclophosphamide, Cytarabine, Daunorubicin, Docetaxel, Doxifluridine, Doxorubicin, Epirubicin, Epothilone, Etoposide, Fluorouracil, Gemcitabine, Hydroxyurea, Idarubicin, Imatinib, Irinotecan, Mechlorethamine, Mercaptopurine, Methotrexate, Mitoxantrone, Oxaliplatin, Paclitaxel, Pemetrexed, Teniposide, Tioguanine, Topotecan, Valrubicin, Vinblastine, Vincristine, Vindesine, and Vinorelbine.

Methods of assessing cell viability are well known to the person skilled in the art. For example, ATP production can be measured, or the incorporation of propidium iodide.

Following treatment with a antineoplastic treatment, any residual cells can be tested for sensitivity to a second antineoplastic treatment.

Thus the method may further comprise assessing residual cancer stem cell sensitivity after initial treatment with a first anti-neoplastic treatment by
(i) isolating neoplastic stem cells based on cell surface marker combinations;
(ii) reaggregating isolated neoplastic stem cells into 3D tissue ; and
(iii) contacting the aggregated neoplastic stem cells with a second anti-neoplasm treatment.

Any neoplastic stem cells remaining in the aggregate following treatment can be identified based on cell surface marker combinations, for example, using flowing cytometry. Cell-surface marker combinations which can be used to identify neoplastic stem cells are known in the art. For example glioblastoma multiforme cancer stem cell markers include PROMININ-1/CD133, SSEA1/CD15, NESTIN, SOX2, BMI1, and MUSASHI. For solid NSCLC tumours, examples of suitable markers include CD31-APC Cy7, CD44-FITC, CD45-PerCp, CD90-BV421, and EpCam-APC.

The neoplastic stem cells present can be isolated and then used to form a new 3D tissue aggregate using the methods described above. It may be necessary to add additional mesenchymal cells in order for the aggregate to form. The aggregate formed from the neoplastic stem cells can then be tested using a different antineoplastic treatment. Thus, the optimal treatment for the patient can be identified so that all of the neoplasm can be targeted.

The term “comprises” or “comprising” or “including” are to be construed here as having a non-exhaustive meaning and allow the addition or involvement of further features or method steps or components to anything which comprises the listed features or method steps or components. “Comprising” can be substituted by “including” if the practice of a given language variant so requires or can be limited to “consisting essentially of” if other members or components are not essential to reduce the invention to practice.

In the present specification, unless indicated otherwise, the singular form of words includes, as to their meaning, the plural form thereof. As used herein the singular forms “a” and “an” before a noun include plural references unless the context indicates otherwise. Any reference to “or” herein is intended to encompass “and/or” unless stated otherwise.

### Exemplary Methods of the Invention.

Suitable methods for processing the neoplastic tissue specimens are described below.

1. Resected Solid Tissue Specimen
1.1. Tumour, metastatic lymph node or/and normal autologous tissue dissociation
   1.1.1. The tumour (and normal tissue if available) sample is obtained from the patient by surgery. If necessary, samples can be stored overnight at 4° C. or even room temperature (up to 20° C.) until processed. Tissue weighing in a range of 0.01-1 g is used for dissociation.
   1.1.2.Wash the tissue minimum of 3-5 times, for example in sterile buffer e.g. phosphate buffer saline (PBS, pH:7.2), to reduce the number of red blood cells
   1.1.3. Mince the tissue with 2 sterile scalpels if necessary
   1.1.4. Prepare the digestion, for example according to the Miltenyi Tumour Dissociation Kit manual using gentle-MACS (using a protocol selected for the specific tumour type). Place the tube into the heated dissociator. For example in case of a lung sample, complete the 37° C._h_TDK_2 (60 min.) and 37° C._m_LDK program (30 min).
   1.1.5. Wash the resulting cell suspensions, for example in sterile PBS and lyse red blood cells if necessary. Methods for lysing red blood cells are known in the art.
   1.1.6. After the digestion is completed, count the cells for further processing.

2. Malignant Pleural or Ascites Fluid
2.1. The volume of drained pleural effusion varies between 200 ml-2500 ml. The appearance in half of the malignant pleural effusion (MPE) is haemorrhagic and bloody in nature. The amount of red blood cells in MPE varies from patient to patient. The volume of ascites fluid ranges between 200 ml-6000 ml (or even above).
2.2. Heparinized samples (1 ml of 1:1000 heparin per 50 ml of pleural fluid) should be submitted for analysis if the pleural fluid is bloody. Samples should be refrigerated e.g. 0-4° C. if not processed within one hour of collection. Cells from MPE are frequently used for pathological evaluation. Sedimented cells from MPE can be used to prepare blocks for cytology by pathologists and differentiate amongst tumour types as e.g. actively dividing mesothelial cells can mimic an adenocarcinoma that is most likely to produce MPE in the first place.
2.3. Processing MPE and ascites fluids
   2.3.1.MPE or ascites fluids are collected usually during surgery (volume varies individually)
   2.3.2. Spin MPE or ascites fluids in closed containers (300 g, 20 min, 4° C.) to sediment cells
   2.3.3. Remove supernatant and re-suspend pellet in the appropriate volume of buffer such as PBS optionally containing 20% of cell free MPE or ascites fluid
   2.3.4 Separate mononuclear cells for example using Ficoll. In this method, Ficoll within conical tubes is overlaid with cell suspension before centrifugation for example at 400 g, for 30 min, at room temperature (about 20° C).
   2.3.4.Any red blood cells are discarded and the remaining cells isolated.
   2.3.5. Re-suspend cells in a suitable buffer e.g. PBS and spin at 400 g, 10 min, at room temperature (about 20° C.) .Preferably the ratio of cells to buffer is 1:3.
   2.3.6. Wash cells in a suitable buffer with centrifugation between washes. For example the cells may be suspended in 50 ml PBS 3× including a spinning step between washes (200 g, 10 min, 4° C.)
   2.3.7.Re-suspend the final pellet in the appropriate volume of buffer such as PBS
   2.3.8.Count cells before further processing

3. Protocols Shared by Both Solid Tissue and Fluid Samples
3.1. Flow cytometry analysis
   3.1.1. Count the cells, spin (for example 200 g, 10 min, 4° C.) then re-suspend in 1 ml buffer e.g. PBS and divide the samples in the necessary number of tubes.
   3.1.2. After another spinning step (as above), discard the supernatant and add 50 μl buffer (PBS)/tube. Identify cell within population for example utilising labelled antibodies specific to known surface cells markers. For example in case of solid NSCLC tumour tissue sample: Stain 0.25-110⁶ cells per test tube with 5 labelled antibodies to detect tumour cell population. The list of antibodies included but not exclusive to: CD31-APC Cy7, CD44-FITC, CD45-PerCp, CD90-BV421, EpCam-APC.
   3.1.3. Incubate the samples for 30 minutes in dark and wash the cells in 1 ml buffer (PBS).
   3.1.4. After a gentle spin remove supernatant and fix the cells e.g. using 300 μl of 1% PFA (paraformaldehyde in PBS).
3.2. Cryo Preservation
   3.2.1. Freeze 1-2×10⁶cells/cryovial from each sample.
   3.2.2. Use “tumour type specific medium” supplemented with DMSO at a final concentration of 10% or directly Cryo-SFM medium (Promocell). Suitable tumour type specific media are known to the skilled person and available commercially e.g. Cancer Stem Cell Media Premium (Promab), Celprogen culture media, etc.
3.3. Reduction of CD45+ cell number

Methods of removing CD45+ cells are known in the art, and kits are commercially available (e.g. Miltenyi; Dynabeads; MagnisortTM). Suitable methods are described below:
3.3.1. Removal of CD45+ cells by ImmunoMagnetic Separation (Miltenyi or EasySep)
   Using the EasySep protocol: Red blood cell free cell suspension is incubated in the presence of Tetrameric Antibody Complexes recognizing CD45 and dextran-coated magnetic particles. Labelled cells are separated using an EasySep™ magnet. Unwanted CD45+ cells remain in the tube over the magnet, while desired cells are poured off for further processing.
3.3.2. Removal of CD45+ cells by Cell Sorter
   Red blood cell free cell suspension is incubated in the presence of FITC-labelled CD45 antibody. The cell suspension is separated from cell debris and dead cells using forward and side scatter. Viable population is gated and FITC+ cells will be visible in the F1 channel. Cells will be collected into two tubes. FITC+ cells will be collected separately while FITC-cells will be processed further.
3.3.3. CD45 positivity will be assessed on the non-lymphoid cell pool and if it is disproportionately too low (<5%), then CD45+ cells can be added back to the suspension.
3.4. Preparation of aggregates
   3.4.1. Calculate the number of cells needed based on:
      3.4.1.1. the size (total cell number ranges between 5-30×10⁴) of planned aggregates
      3.4.1.2. the added ratio of fibroblasts (such as Normal Human Lung Fibroblasts)(maximum of 50%) in the aggregates if necessary,
      3.4.1.3. the total number of aggregates (in triplicates) for reliable drug sensitivity analysis
   3.4.2. Prepare mixed cell suspension according to the above calculation and supplement with the adequate volume of suitable tumour type specific media e.g.“Lung tumour medium” (the total volume should be 200 μl/spheroid).
   3.4.3. Pipette 200 μl/well mixed suspension into a sterile 96-well, U bottom cell culture plate with Ultra-low attachment surface.
   3.4.4. Fill the empty wells with 200 μl sterile PBS (multichannel pipette can be used for this step).
   3.4.5. Centrifuge the plate at e.g. 600×g for 10 minutes at room temperature.
   3.4.6. Transfer the plate into a 37° C., 5% CO₂, humidified incubator for 24 hours.
3.5. Treatment of 3D aggregates
   3.5.1. An example protocol for an NSCLC sample
   3.5.2. If required, subject to treatment with the anti-neoplasm compound to the wells. Pipette 200 μl/well mixed “Lung tumour medium” with the anti-neoplasm compound (applied concentration) to the wells.
   3.5.3. Example of applied concentrations: Cisplatin: 6 or 9 μg/ml, Erlotinib: 100 nM or 1 μM, Vinorelbine: 20 or 50 nM
   3.5.4.Transfer the plate into a 37° C., 5% CO₂, humidified incubator for 24, 48 or 72 hours.
3.6. Viability assay
   3.6.1.Maintain the spheroids in 200 ul mixed “tumour type specific medium”
   3.6.2.Add equal volume of CellTiter Glo (Promega) reagent, shake it vigorously for 5 minutes and incubate the plate for 25 min at RT
   3.6.3.Measure the viability signal with a luminometer
3.7. Flow cytometry analysis
   3.7.1. Following tests, collect aggregates (minimum of 100 000 cells/treatment is necessary) and wash in PBS
   3.7.2. Disaggregate aggregated tissues using trypsin and collagenase (37° C., 30 min, RT)
   3.7.3. Count the cells, spin (200 g, 10 min, 4° C.) then re-suspend them in the appropriate volume of PBS and divide the samples in the necessary number of tubes.
   3.7.4. After another spinning step (as above), discard the supernatant and add 50 μl PBS/tube. In case of aggregates prepared from solid NSCLC tumour tissue samples: Stain 0.25-110⁵ cells per test tube with 5 labelled antibody to detect tumour cell population. The list of antibodies included but not exclusive to: CD31-APC Cy7, CD44-FITC, CD45-PerCp, CD90-BV421, EpCam-APC.
   3.7.5. Incubate the samples for 30 minutes in dark and wash the cells in 1 ml PBS. 3.7.6. After a gentle spin remove supernatant and fix the cells with 300 μl of 1% PFA
   (paraformaldehyde in PBS).
3.8. Tumour cryovials
   3.8.1. Thawing of cryovials
   3.8.2. Pre-warm a 37° C. water bath and thaw the cryovials for no longer than 2 minutes.
   3.8.3. Dispense the cells in a 50 ml tube and slowly (drop by drop) pipette 20 ml of pre-warmed complete cell culture medium to the cells.
   3.8.4. Centrifuge e.g. 5 minutes at 200 g.
   3.8.5. Repeat step 3.8.3 once again.
   3.8.6.Re-suspend the pellet in 1 ml of “tumour specific medium” and count cells for further application.

The invention will now be described with reference to the following examples with refer to the following figures:

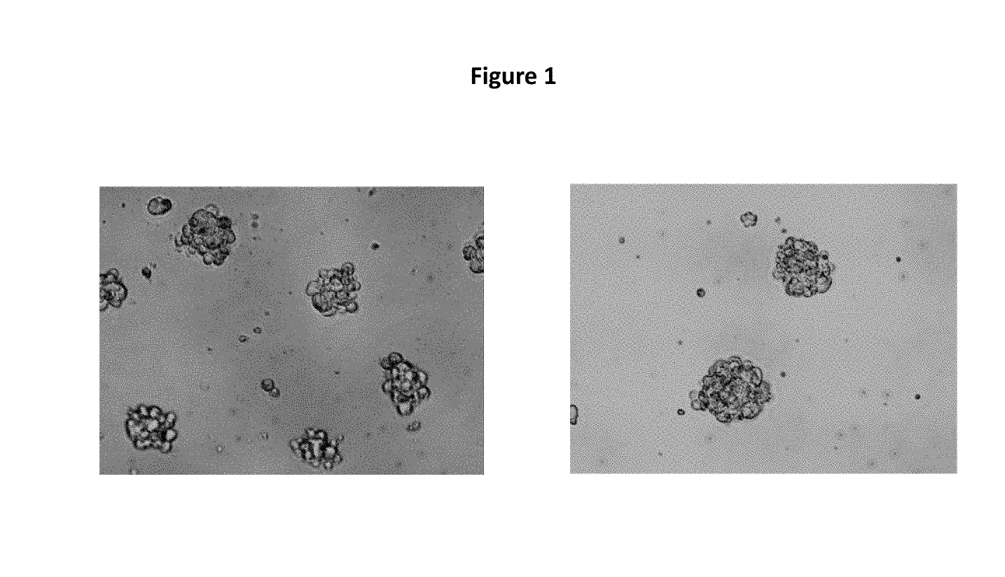

FIG. 1 shows Glioblastoma multiforme “out-growth” cultures.

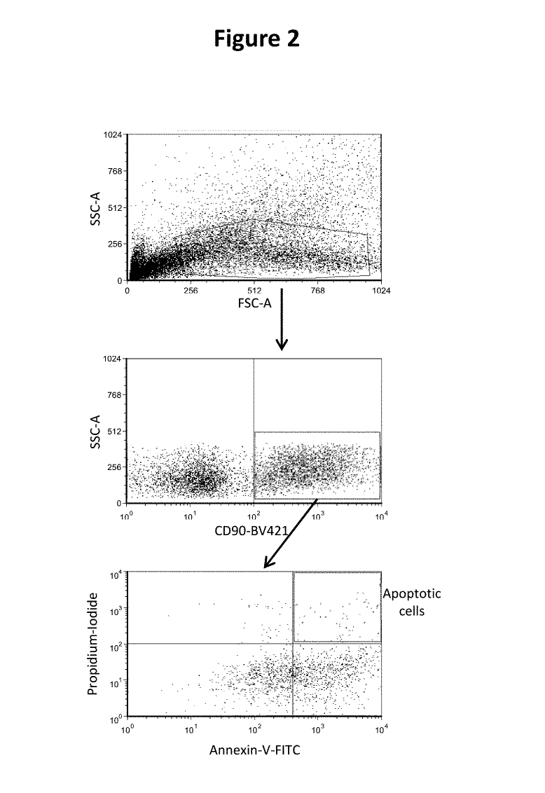

FIG. 2 shows the results of flow cytometric analysis of glioblastoma multiforme.

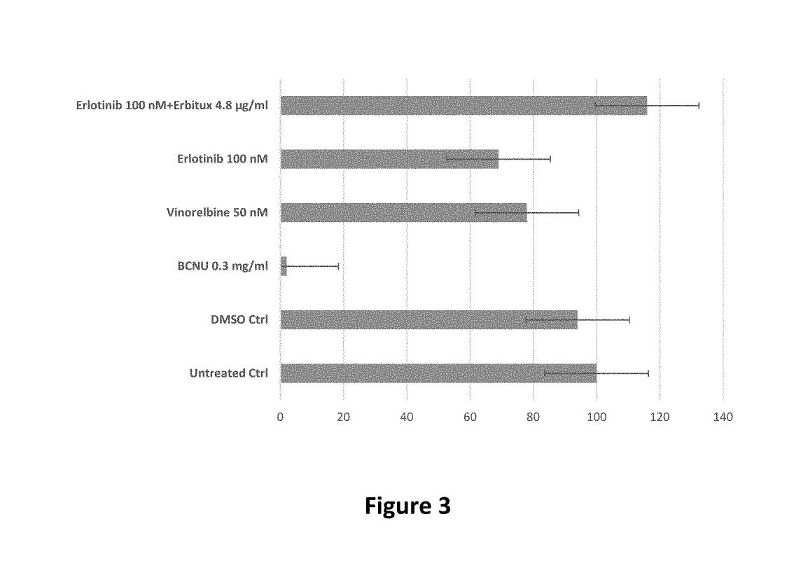

FIG. 3 shows the response of Glioblastoma multiforme 3D aggregates after 72 hr incubation with various drugs.

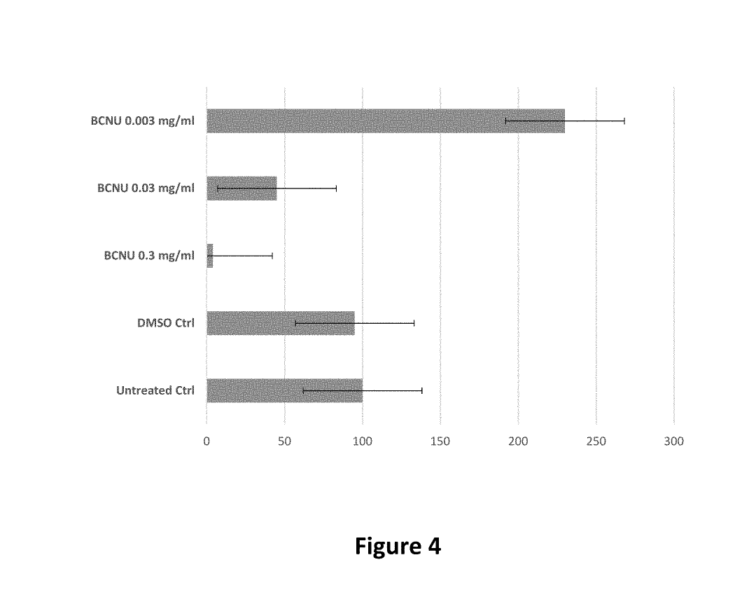

FIG. 4 shows the response of Glioblastoma multiforme 3D aggregates after 24 hr incubation with different concentrations of BCNU.

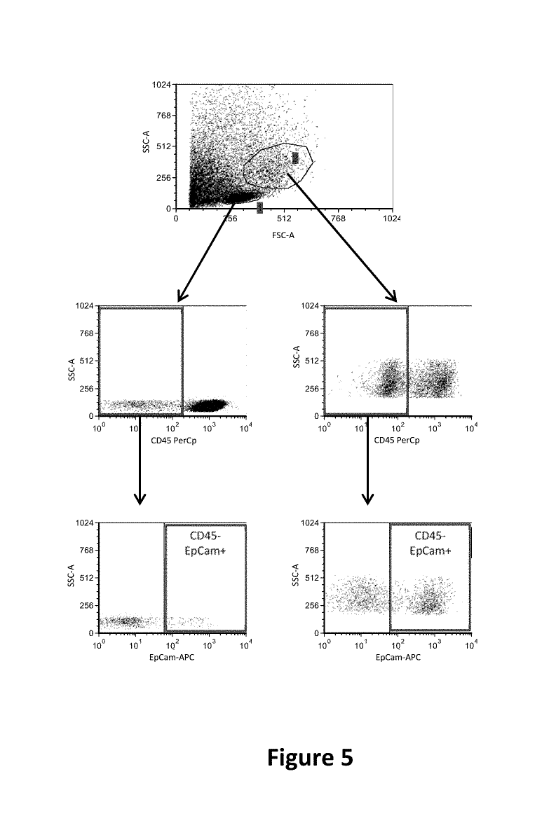

FIG. 5 shows the results of flow cytometric analysis of adenocarcinoma pulmonis.

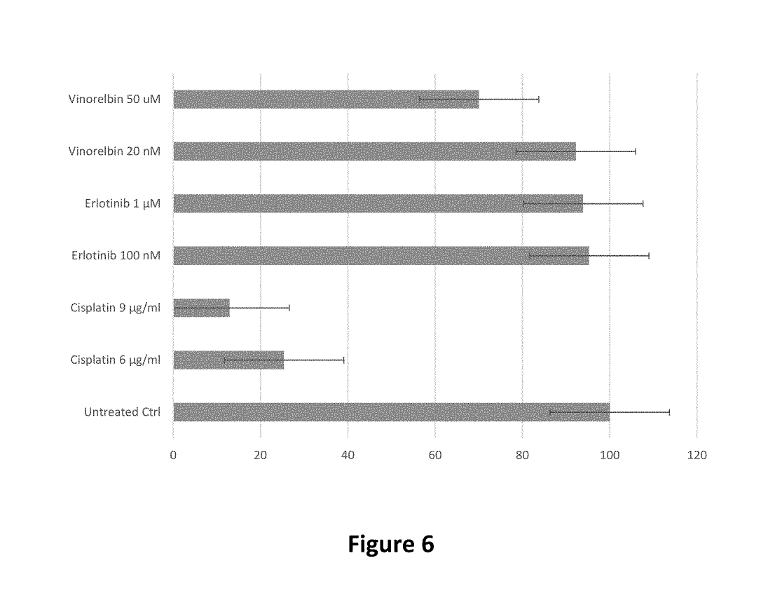

FIG. 6 shows the response of NSCLC Adenocarcinoma 3D aggregates after 72 hr incubation with different concentrations of monotherapies.

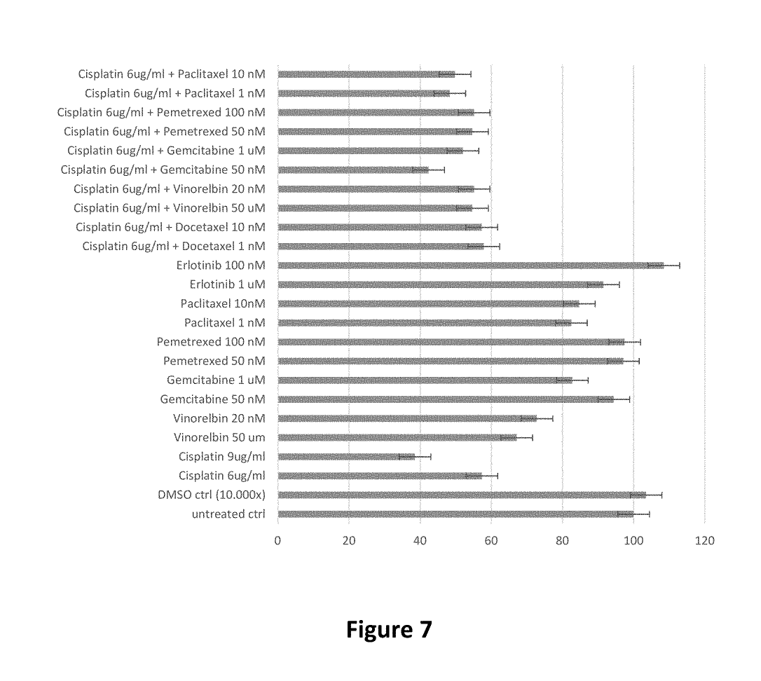

FIG. 7 shows the response of Testicular cancer 3D aggregates after 48 hr incubation with different concentrations and different combinations of drugs.

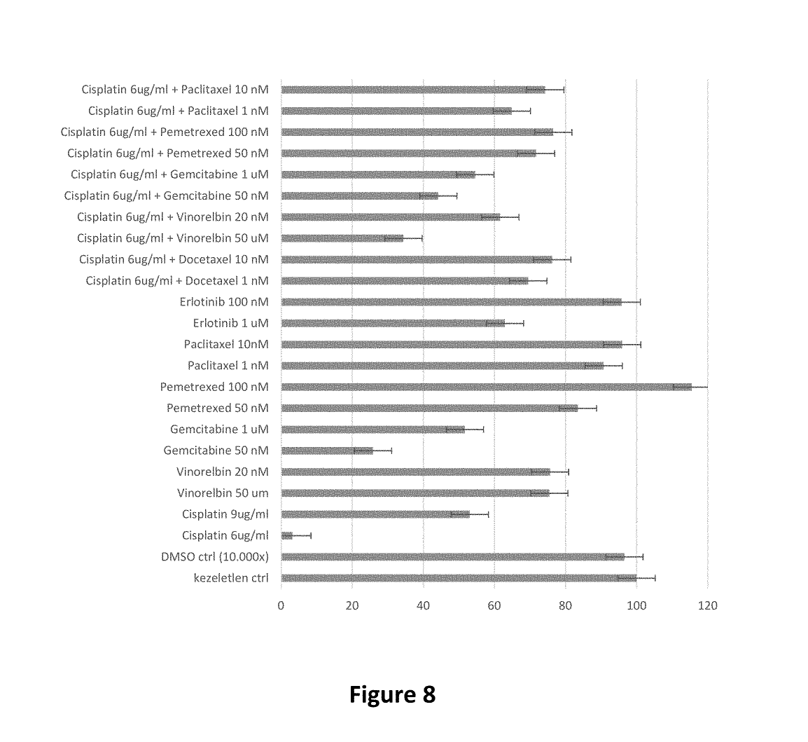

FIG. 8 shows the response of Malignant pleural fluid cells 3D aggregates after 48 hr incubation with different concentrations and different combinations of drugs

### EXAMPLES

1. Solid Tumour

1.1 Primary Glioblastoma

Glioblastoma multiforme is one of the deadliest of neoplasms and continues to be regarded as incurable and universally fatal. This reputation seems well deserved, based on population-based outcome data from multiple centres over decades of investigation. Only a couple of percent of glioblastoma patients survive three years or longer, and five-year survival is still exceptionally rare.

Glioblastoma Multiforme Drug Sensitivity Analysis

Two, freshly resected, native samples reached the laboratory directly from the pathologist within 2 hours of surgery. The two samples were treated separately and were labelled as “Glioblastoma 1” and “Glioblastoma 2”. The pathologist identified the macroscopically identical tumour samples as Glioblastoma 1 (Sample 1) being fully viable while Glioblastoma 2 (sample 2) as strongly necrotic. The samples were processed according to protocol and drug sensitivity tests were performed using the viable, Sample 1. Samples for DNA and RNA isolation were also stored at −80° C., leaving the opportunity open for additional sequencing or comparative gene expression studies. Traditional out-growth cultures were also prepared from Glioblastoma sample 1 showing the strong viability and proliferative ability of the cells (FIG. 1.).

Analysis Methods:

Toxicology assay: CellTiter-Glo® 3D Cell Viability Assay (Promega). The CellTiter-Glo® 3D Cell Viability Assay is a homogeneous, luminescent method to determine the number of viable cells in 3D cell culture based on quantitation of the ATP present, which is a marker for the presence of metabolically active cells.

Annexin: Annexin V is used as a non-quantitative probe to detect cells that express phosphatidylserine (PS) on their cell surface, an event found in apoptosis as well as other forms of cell death. The assay combines annexin V staining of PS and PE membrane events with the staining of DNA in the cell nucleus with propidium iodide (PI) or 7-Aminoactinomycin D (AAD-7), distinguishing viable cells from apoptotic cells and necrotic cells. Detection was performed by flow cytometry or a fluorescence microscope.

Cellular markers: GBM cancer stem cell markers: PROMININ-1/CD133, SSEA1/CD15, NESTIN, SOX2, BMI1, MUSASHI. Analysis is performed using flow cytometry and cytospin/tissue section staining and fluorescence microscopy (FIG. 2).

Drug Sensitivity Test

Aggregates were prepared in 96-well plates and cultures were incubated with the following agents: cisplatin, erlotinib, vinorelbine, and pemetrexed. 4 wells/treatment were tested, aggregates were cultured for 24, 48 or 72 h respectively, at 37° C. using the drugs in concentrations as: Cisplatin: 6 or 9 μg/ml, Erlotinib (Tarceva): 100 nM or 1 μM, Vinorelbine (Vinorelbine is a drug acting by a similar mechanism to Vincristine frequently used in neurooncology): 20 or 50 nM; Erbitux (Cetuximab): 4.8 mg/ml; BCNU (Carmustine): 0.3 mg/ml, 0.03 mg/ml, 0.003 mg/ml. Erlotinib (Tarceva) +Erbitux. Erlotinib similarly to Cetuximab is an EGFR inhibitor (the two drugs are frequently used clinically together). Following 24, 48 h or 72 h incubation, cells were labelled using Annexin V-PI and analyzed by flow cytometry or analysed by Promega CellTiter-Glo® 3D Cell Viability Assay Kit (Luminescent) (ATP detection kit)(FIGS. 3 & 4).

Glioblastoma 1

| | Annexin Pl++ | Pl+ ratio |
|---|---|---|
| | Late | within Annexin+ |
| | apoptosis | Non-viable |
| | % | % |
| SD | 0.50 | 0.94 |
| Cisplatin 9 μg/m1 | 0.19 | 0.44 |
| Erlotinib 1 μM | 0.32 | 0.62 |
| Pemetrexed 1 μM | 0.19 | 0.37 |
| Vinorelbine 20 nM | 0.49 | 0.93 |
| Vinorelbine 50 nM | 0.05 | 0.09 |

The results clearly confirmed sensitivity of the glioblastoma cells to BCNU. The patient was treated with BCNU and the tumour was regressing within 2 weeks after the first administration of the drug.

1.2 Non-Small Cell Lung Cancer

Eighty percent of all diagnosed lung cancers are non-small cell lung cancer. The 5-year survival rate of NSCLC varies from 73% in early detection (stage IA) to 3.7% at advanced metastatic disease. At early stages of NSCLC surgery and chemotherapy are still the choice of first line treatment, while in metastatic disease the focus is on chemotherapy.

NSCLC Drug Sensitivity Analysis

Freshly resected native lung carcinoma sample reached our laboratory within 24 h of surgery. Diagnosis was confirmed as NSCLC, adenocarcinoma pulmonis, (predominantly acinar, with a 30% lepidic component) pT1b N1. PN+, LI−, R0.

Analysis Methods:

Toxicology assay: CellTiter-Glo® 3D Cell Viability Assay (Promega). The CellTiter-Glo® 3D Cell Viability Assay is a homogeneous, luminescent method to determine the number of viable cells in 3D cell culture based on quantitation of the ATP present, which is a marker for the presence of metabolically active cells.

Cellular markers: Analysis is performed using flow cytometry and cytospin/tissue section staining and fluorescence microscopy (FIG. 5).

Drug Sensitivity Test

Aggregates were prepared in 96-well plates and cultures were incubated with the following agents: cisplatin (6 or 9 μg/ml), pemetrexed (50 nM and 100 nM), gemcitabine (50 nM and 1 μM), docetaxel (1 nM and 10 nM), paclitaxel (1 nM and 10 nM) and their clinically applied combinations. 4 wells/treatment were tested, aggregates were cultured for 24, 48 h or 72 h at 37° C. Following incubation cells were analysed by Promega CellTiter-Glo® 3D Cell Viability Assay Kit (Luminescent) (ATP detection kit) (FIG. 6).

The results clearly pointed out the cisplatin+gemcitabine combination as the most successful of chemotherapeutic combinations. The patient was treated with a Cisplatin+Gemcitabine combination and the disease has not been progressing.

1.3 Testicular Cancer

Testicular cancer has one of the highest cure rates of all cancers with an average five-year survival rate of 95%. If the cancer has not spread outside the testicle, the 5-year survival is 99% while if it has grown into nearby structures or has spread to nearby lymph nodes, the rate is 96% and if it has spread to organs or lymph nodes away from the testicles, the 5-year survival is around 74%. Even for the relatively few cases in which cancer has spread widely, chemotherapy offers a cure rate of at least 80%.

Testicular Cancer Drug Sensitivity Analysis

Analysis Methods:

Toxicology assay: CellTiter-Glo® 3D Cell Viability Assay (Promega). The CellTiter-Glo® 3D Cell Viability Assay is a homogeneous, luminescent method to determine the number of viable cells in 3D cell culture based on quantitation of the ATP present, which is a marker for the presence of metabolically active cells.

Drug Sensitivity Test

Aggregates were prepared in 96-well plates and cultures were incubated with the following agents: cisplatin (6 or 9 μg/ml), pemetrexed (50 nM and 100 nM), gemcitabine (50 nM and 1 μM), docetaxel (1 nM and 10 nM), paclitaxel (1 nM and 10 nM) and their clinically applied combinations. 4 wells/treatment were tested, aggregates were cultured for 24, 48 h or 72 h at 37° C.

Following incubation cells were analysed by Promega CellTiter-Glo® 3D Cell Viability Assay Kit (Luminescent) (ATP detection kit) (FIG. 7).

2. Malignant Pleural Fluid

Malignant pleural effusion (MPE) usually presents in the disseminated and advanced stage of malignancy. Dyspnea is the debilitating symptom which needs palliation in these patients. By this stage of the disease there is no cure.

NSCLC Malignant Pleural Fluid Drug Sensitivity Analysis

Thoracentesis was performed on the patient who was presented with dyspnea and no prior diagnosis of neoplasm. Diagnosis was confirmed as NSCLC, adenocarcinoma, T4 Nx. M1.

Analysis Methods:

Toxicology assay: CellTiter-Glo® 3D Cell Viability Assay (Promega). The CellTiter-Glo® 3D Cell Viability Assay is a homogeneous, luminescent method to determine the number of viable cells in 3D cell culture based on quantitation of the ATP present, which is a marker for the presence of metabolically active cells.

Drug Sensitivity Test

Aggregates were prepared in 96-well plates and cultures were incubated with the following agents: cisplatin (6 or 9 μg/ml), pemetrexed (50 nM and 100 nM), gemcitabine (50 nM and 1 μM), docetaxel (1 nM and 10 nM), paclitaxel (1 nM and 10 nM) and their clinically applied combinations. 4 wells/treatment were tested, aggregates were cultured for 24, 48 h or 72 h at 37° C.

Following incubation cells were analysed by Promega CellTiter-Glo® 3D Cell Viability Assay Kit (Luminescent) (ATP detection kit) (FIG. 8).

1. A 3-Dimensional (3D) tissue culture aggregate of cells derived from a neoplastic tissue sample wherein 30% of total number cells are cells capable of interfering with re-aggregation; wherein said aggregate does not contain an artificial scaffold.

2. The 3D tissue culture aggregate of claim 1 wherein the cells capable of interfering with re-aggregation are lymphoid cells.

3. The 3D tissue culture aggregate of claim 1 wherein the cells capable of interfering with re-aggregation are CD45+.

4. A method for preparing a 3D tissue culture aggregate comprising:
(a) Preparing an adjusted cell population from a neoplastic tissue sample by reducing the number of cells capable of interfering with re-aggregation to ≤30% of total number cells; and
(b) Preparing a suspension culture comprising cells of said adjusted cell population, culture media and optionally fibroblasts; in the absence of an artificial scaffold.

5. The method of claim 4 wherein the number of fibroblasts in the initial suspension culture is 5-50% total number of cells.

6. The method of claim 4 wherein the number of cells from the adjusted cell population in the initial suspension culture is 2×104 to 8×106.

7. The method of claim 4 wherein the number of cells capable of interfering with re-aggregation is reduced by an immunological particle separation method or a cell sorting separation method.

8. The method of claim 4 wherein the extracellular matrix in the three dimensional (3D) neoplasm tissue culture aggregates is only produced by the cells themselves.

9. The method of claim 4, wherein the cells capable of interfering with re-aggregation are lymphoid cells.

10. The method of claim 4, wherein the cells capable of interfering with re-aggregation are CD45+.

11. The use of a 3D tissue culture aggregate of claim 1 to assess the effectiveness of an anti-neoplasm treatment.

12. A method for assessing the effectiveness of an anti-neoplasm treatment by measuring the effect of said treatment on the viability of a three dimensional (3D) neoplasm tissue culture aggregates.

13. The method of claim 12 wherein said 3D neoplasm tissue culture aggregates is a 3D tissue culture aggregate of claim 1.

14. The method of claim 12 wherein the viability of 3D neoplasm tissue culture aggregates is measured by using a cell viability assay.

15. The method of claim 12 further comprising determining the cellular composition of the 3D neoplasm tissue culture aggregates by cell surface marker analysis using flow cytometry.

16. The method of claim 12 further comprising assessing residual cancer stem cell drug sensitivity after a first anti-neoplastic agent treatment by
(i) isolating neoplastic stem cells based on cell surface marker combinations;
(ii) reaggregating isolated neoplastic stem cells into 3D tissue; and
(iii) contacting the aggregated neoplastic stem cells with a second anti-neoplastic treatment, wherein said first antineoplastic treatment and said second antineoplastic treatment are different.

17. The 3D tissue culture aggregate of claim 2 wherein the cells capable of interfering with re-aggregation are CD45+.

18. The method of claim 5 wherein the number of cells from the adjusted cell population in the initial suspension culture is 2×104 to 8×106.

19. The method of claim 5 wherein the number of cells capable of interfering with re-aggregation is reduced by an immunological particle separation method or a cell sorting separation method.

20. The method of claim 6 wherein the number of cells capable of interfering with re-aggregation is reduced by an immunological particle separation method or a cell sorting separation method.
